(19) United States
(12) Patent Application Publication
(10) Pub. No.: US 2026/0245210 A1
(43) Pub. Date: Aug. 20, 2026

(54) IMAGE DATA-BASED MUSCULOSKELETAL STATUS ANALYSIS SYSTEM

(71) Applicant: MG Solutions Co., Ltd., Seoul (KR)

(72) Inventors: Hyun Jun KIM, Seoul (KR); Jae Hyun PARK, Seoul (KR)

(21) Appl. No.: 19/245,781

(22) Filed: Jun. 23, 2025

(30) Foreign Application Priority Data
Feb. 17, 2025 (KR) 10-2025-0020326

(51) Int. Cl. G06T 7/00; A61B 5/00; G06T 7/194; G06T 7/50; G06T 7/73; G16H 50/30

(52) U.S. Cl. CPC G06T 7/0014; A61B 5/4538; A61B 5/742; G06T 7/194; G06T 7/50; G06T 7/73; G16H 50/30; G06T 2207/10028; G06T 2207/20084; G06T 2207/30008

(57) ABSTRACT

The present disclosure relates to an image data-based musculoskeletal status analysis system which accurately checks a skeleton and a muscular condition of a subject to be measured by finding out skeleton points a body of a subject to be measured using an image processing technology, and assists correction based on checked results. The image data-based musculoskeletal status analysis system includes: an image processing part configured to receive front image data, lateral image data, and back image data of a subject to be measured, and extract depth frame data of the subject to be measured from the front image data, the lateral image data, and the back image data, respectively; a skeleton point extraction part configured to receive the pieces of depth frame data of the subject to be measured from the image processing part and calculate the pieces of depth frame data of the subject to be measured to extract pieces of skeleton point data; an evaluation item calculation part configured to calculate values of evaluation items for evaluating a skeletal status from the pieces of skeleton point data extracted by the skeleton point extraction part; and a muscular condition diagnosis part configured to diagnose a muscular condition by comparing the values of the evaluation items calculated by the evaluation item calculation part with data in a normal state.

### TECHNICAL FIELD

The present disclosure relates to a system for capturing an image of a subject to be measured with a camera to analyze a musculoskeletal status of the subject to be measured, and more particularly to an image data-based musculoskeletal status analysis system which is capable of finding skeleton points of a body of a subject to be measured using an image processing technology to accurately check a musculoskeletal status of the subject to be measured, and assisting correction based on the checked results.

### BACKGROUND

In recent years, many people are rapidly becoming obese and their body type is also becoming increasingly unbalanced due to lack of exercise. Such an imbalance in body type may cause various diseases. As the imbalance in body type becomes severe, body-type diseases such as pelvic imbalance, knee flexion, imbalance growth of muscles around the knee, spinal deformations such as a turtle neck and straight neck, spine-pelvis curvature, instability of thoracic muscle and ligament tissue, back knee, pelvic-leg imbalance, imbalance growth of back muscles of the knee, and spine (skull, spine or the like) curvature are likely to occur.

A body-type analysis needs to prevent such body-type diseases. Korean Patent Application Publication No. 10-2010-0092555 (hereinafter, referred to as “Patent Document 1”), entitled “Body-type analysis device using Web camera”, discloses a technology which analyzes a body type using a motor capable of moving the Web camera up and down, a personal computer capable of acquiring image data taken by the Web camera, and an algorithm for quantitatively automatically analyzing position values of a marker in an image and an inclination of the body.

However, since the technology disclosed in Patent Document 1 is a method of attaching the marker to the body, it may cause inconvenience to use. In addition, since the Web camera connected to a belt moves up and down by an electric motor, analysis results may not be accurate.

Therefore, in order to accurately diagnose a musculoskeletal status, a skeletal status of a subject to be measured who stands naturally needs to be captured by using X-ray. In general, diagnosis capturing is performed by a radiologist. In order to obtain a full-body photograph using a general-purpose X-ray device and check a skeletal status of the full body, it is necessary to capture an image of a front body portion 4 to 5 times and also capture an image of a lateral body portion 4 to 5 times. Accordingly, when such a general-purpose X-ray device is used to capture the full body of the subject to be measured, the subject to be measured may be exposed excessively to radiation.

Further, rehabilitation treatment for a musculoskeletal system is being widely applied to various healthcare centers, such as general fitness clubs, Pilates and Yoga, as well as schools and hospitals. However, the X-ray device as a medical equipment cannot be used in such healthcare centers. As a result, services such as body correction and spinal correction may be provided to members of the centers without a proper diagnosis. Therefore, there is a need to provide an apparatus capable of accurately analyzing a musculoskeletal status of a full body of a subject to be measured.

To achieve this, the present applicant(s) proposed a big data-based musculoskeletal analysis apparatus (Korean Patent Application Registration No. 10-2355184 (hereinafter, referred to as “Patent Document 2”). The musculoskeletal analysis apparatus disclosed in Patent Document 2 is capable of extracting skeleton points and checking a muscular condition of a subject to be measured based on the extracted skeleton points, which eliminates a need to use radiation, markers or the like. However, in the musculoskeletal analysis apparatus disclosed in Patent Document 2, a process of extracting the skeleton points is somewhat complicated.

### SUMMARY

The present disclosure was made to solve the above-mentioned matters, and the present disclosure is for the purpose of providing a musculoskeletal status analysis system capable of finding skeleton points of a body from an image of a subject to be measured to evaluate a muscular condition and accurately providing musculoskeletal status of the subject to be measured.

Further, the present disclosure is for the purpose of providing a musculoskeletal status analysis system capable of extracting depth frame data from an image of a subject to be measured, extracting data about skeleton points of a body of the subject to be measured from the depth frame data, and extracting the skeleton points at an improved accuracy while simplifying a process of extracting the skeleton points of the body.

An image data-based musculoskeletal status analysis system according to an example embodiment of the present disclosure for solving the above-described matters may include: an image processing part configured to receive front image data, lateral image data, and back image data of a subject to be measured, and extract depth frame data of the subject to be measured from the front image data, the lateral image data, and the back image data, respectively; a skeleton point extraction part configured to receive the pieces of depth frame data of the subject to be measured from the image processing part and calculate the pieces of depth frame data of the subject to be measured to extract pieces of skeleton point data; an evaluation item calculation part configured to calculate values of evaluation items for evaluating a skeletal status from the pieces of skeleton point data extracted by the skeleton point extraction part; and a muscular condition diagnosis part configured to diagnose a muscular condition by comparing the values of the evaluation items calculated by the evaluation item calculation part with data in a normal state.

In an aspect, the image processing part may be configured to: extract front depth frame data, lateral depth frame data, and back depth frame data from the front image data, the lateral image data, and the back image data, respectively; and remove background depth frame data included in each of the front depth frame data, the lateral depth frame data, and the back depth frame data to extract the depth frame data of the subject to be measured.

In an aspect, the skeleton point extraction part may be configured to classify front body frame data, lateral body frame data, and back body frame data from the depth frame data of the subject to be measured through a superpixel algorithm.

In an aspect, the skeleton point extraction part may be configured to extract front body portion data, lateral body portion data and back body portion data from the front body frame data, the lateral body frame data and the back body frame data through a convolutional neural network (CNN) operation.

In an aspect, the skeleton point extraction part may be configured to calculate center points included in the front body portion data, the lateral body portion data, and the back body portion data to extract front skeleton point data, lateral skeleton point data, and back skeleton point data, respectively.

In an aspect, the skeleton point extraction part is configured to extract the front skeleton point data, the lateral skeleton point data, and the back skeleton point data which include coordinates on X, Y, and Z axes through

$${C}_{x} = {\frac{1}{\vert R \vert} \sum x}_{( x , y , z ) ∈ R} , {C}_{y} = {\frac{1}{\vert R \vert} \sum y}_{( x , y , z ) ∈ R} , {C}_{z} = {\frac{1}{\vert R \vert} \sum z}_{( x , y , z ) ∈ R}$$

(where “C” represents the center point, and “R” represents all pixels classified by a body portion of the subject to be measured from the depth frame data of the subject to be measured).

In an aspect, the skeleton point extraction part may be configured to extract: the front skeleton point data including coordinates of a center of a cranial bone on the X, Y, and Z axes, coordinates of a vertebral body centroid of a seventh cervical vertebra on the X, Y, and Z axes, coordinates of a left acromion portion on the X, Y, and Z axes, coordinates of a right acromion portion on the X, Y, and Z axes, coordinates of a middle portion of a manubrium on the X, Y, and Z axes, coordinates of a left femur on the X, Y, and Z axes, coordinates of a right femur on the X, Y, and Z axes, coordinates of a left patellar on the X, Y, and Z axes, coordinates of a right patellar on the X, Y, and Z axes, coordinates of a left talus on the X, Y, and Z axes, coordinates of a right talus on the X, Y, and Z axes, and coordinates of a center of gravity (CoG) of the subject to be measured on the X, Y, and Z axes; the lateral skeleton point data including the coordinates of the center of the cranial bone on the X, Y, and Z axes, the coordinates of the vertebral body centroid of the seventh cervical vertebra on the X, Y, and Z axes, coordinates of a spinous process of the seventh cervical vertebra on the X, Y, and Z axes, coordinates of a greater tubercle of humerus on the X, Y, and Z axes, coordinates of centroids of first to twelfth vertebral bodies on the X, Y, and Z axes, coordinates of centroids of thirteenth to seventeenth vertebral bodies on the X, Y, and Z axes, the coordinates of the left femur on the X, Y, and Z axes, the coordinates of the left patellar on the X, Y, and Z axes, and the coordinates of the left talus on the X, Y, and Z axes; and the back skeleton point data including the coordinates of the centroids of the first to twelfth vertebral bodies on the X, Y, and Z axes, and the coordinates of centroids of the thirteenth to seventeenth vertebral bodies on the X, Y, and Z axes.

In an aspect, the evaluation item calculation part may be configured to calculate evaluation items including an asymmetrical shoulder height, a head posture, a round shoulder, a scoliosis, a thoracic kyphosis, a straight back syndrome, a lumbar lordosis, a flat back syndrome, a pelvic tilt, a pelvic axial rotation, a pelvic obliquity, a Hip-Knee-Axis (HKA) angle left, a HKA angle right, a knee flexion, and a back knee.

In an aspect, the image data-based musculoskeletal status analysis system may further include: a skeleton simulation part configured to generate data for displaying a skeletal morphology of the subject to be measured based on the pieces of skeleton point data of the subject to be measured and the values of the evaluation items; an evaluation result comparison part configured to compare result values calculated by the evaluation item calculation part with normal state data of a normal state data part; a risk rate prediction part configured to predict a risk rate based on a comparison result obtained by the evaluation result comparison part and provide prediction information; and a user interface part configured to provide skeleton simulation data obtained by the skeleton simulation part, data on the muscular condition obtained by the muscular condition diagnosis part, and the prediction information obtained by the risk rate prediction part to a user via a display part or a printer.

According to the present disclosure, it is possible to capture an image of a subject to be measured who stands naturally without having to attach a marker such as a marker or the like to a body of the subject to be measured, which provides usability.

Further, according to the present disclosure, it is possible to accurately analyze a musculoskeletal status of the subject to be measured.

Further, according to the present disclosure, it is possible to display measurement results of the subject to be measured in-situ in various healthcare centers in an easy-to-understand manner with graphics, letters, or the like, which allows a user to directly check analysis results and enables an efficient correction.

### BRIEF DESCRIPTION OF THE DRAWINGS

FIG. 1 is a view schematically illustrating a musculoskeletal status analysis system according to an example embodiment of the present disclosure.

FIG. 2 is a block diagram illustrating a main configuration of a musculoskeletal status analysis system according to example embodiments of the present disclosure.

FIGS. 3A to 3C are views for explaining front image data, depth frame data, and depth frame data of a subject to be measured according to an example embodiment of the present disclosure.

FIGS. 4A to 4C are views for explaining lateral image data, depth frame data, and depth frame data of the subject to be measured according to an example embodiment of the present disclosure.

FIGS. 5A to 5C are views for explaining back image data, depth frame data, and depth frame data of the subject to be measured according to an example embodiment of the present disclosure.

FIGS. 6A to 6C are views for explaining Adams image data, depth frame data, and depth frame data of the subject to be measured according to an example embodiment of the present disclosure.

FIGS. 7A and 7B as views for explaining a superpixel algorithm according to an example embodiment of the present disclosure.

FIGS. 8A to 8C are views for explaining skeleton point data on a front side, a lateral side, and a back side according to an example embodiment of the present disclosure.

FIG. 9 is a flowchart for explaining a musculoskeletal analysis procedure according to an example embodiment of the present disclosure.

FIG. 10 is a sequence chart illustrating a procedure of updating reference data by big data analysis according to an example embodiment of the present disclosure.

### DETAILED DESCRIPTION

The present disclosure and technical matters to be solved by the carrying-out of the present disclosure will become more apparent by the preferred example embodiments of the present disclosure which will be described later. The following example embodiments will be merely described to explain the present disclosure, and are not intended to limit the scope of the present disclosure. Hereinafter, example embodiments according to the technical sprit of the present disclosure will be described with reference to the accompanying drawings.

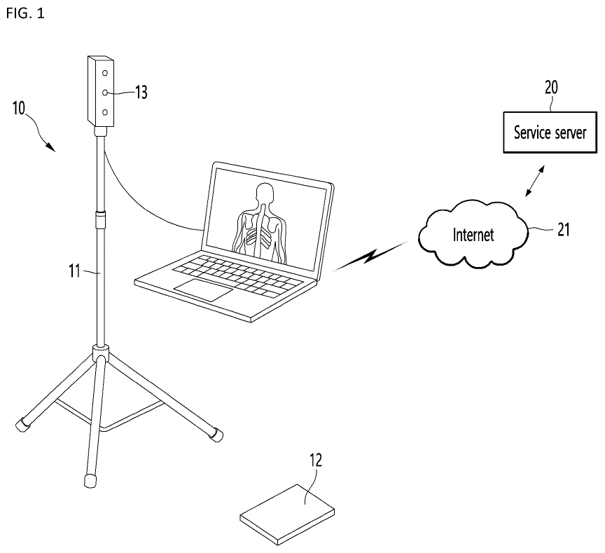

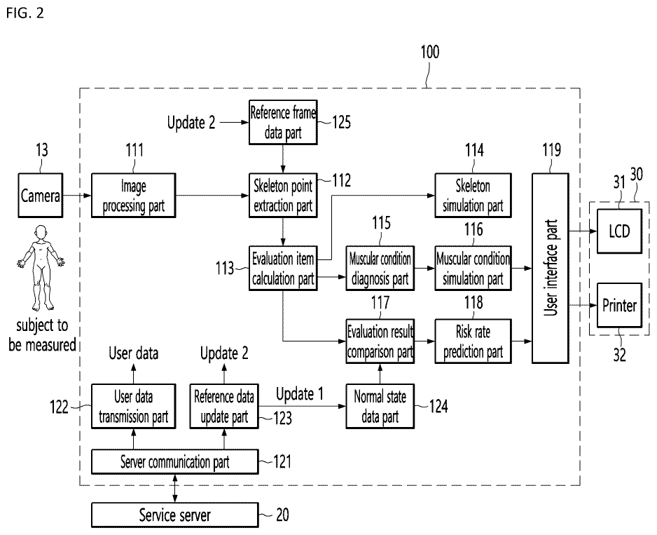

FIG. 1 is a view schematically illustrating a musculoskeletal status analysis system according to an example embodiment of the present disclosure. FIG. 2 is a block diagram illustrating a main configuration of the musculoskeletal status analysis system according to example embodiments of the present disclosure.

Referring to FIGS. 1 and 2, a musculoskeletal status analysis system 10 according to an example embodiment of the present disclosure includes a frame 11, a footplate 12 on which a subject to be measured stands for measurement, a camera 13 for capturing an image of the subject to be measured, a musculoskeletal status analysis system 100 for analyzing image data about the image captured by the camera 13 to analyze a musculoskeletal status of the subject to be measured, and a display 31 for providing the analyzed image data. The musculoskeletal status analysis system 100 constitutes a main body of the musculoskeletal status analysis system 10, and is connected to a service server 20 configured to provide reference data for a big data-based analysis, via a network 21.

The frame 11 is a mechanical device for supporting the display 31, the musculoskeletal status analysis system 100, the camera 13, and the like. The footplate 12 is disposed at a certain position in front of the camera 13. A foot-like position may be displayed on the footplate 12 to accurately guide a position of the subject to be measured during measurement. The camera 13 may capture the image of the subject to be measured who stands naturally and doesn't have a marker or the like, and provide data about the captured image to the musculoskeletal status analysis system 100. The musculoskeletal status analysis system 100 may be implemented with a predetermined computer program.

The musculoskeletal status analysis system 100 of this example embodiment includes an image processing part 111, a skeleton point extraction part 112, an evaluation item calculation part 113, a skeleton simulation part 114, a muscular condition diagnosis part 115, a muscular condition simulation part 116, an evaluation result comparison part 117, a risk rate prediction part 118, a user interface part 119, a server communication part 121, a user data transmission part 122, a reference data update part 123, a normal state data part 124, and a reference frame data part 125. A control part controls an overall operation by executing software according to an operation made by an input part such as a keyboard, a mouse, or the like, and outputs musculoskeletal analysis results to the display 31, a printer 32, and the like of a result display part 30.

The image processing part 111 is configured to receive the image data obtained by the camera 13 and obtain depth frame data of the subject to be measured based on the image data.

The image processing part 111 receives front image data, lateral image data, and back image data of the subject to be measured from the camera 13. The front image data, the lateral image data, and the back image data may be color frame data, and may refer to as photograph data represented by red-green-blue (RGB).

For reference, the back image data may include Adams test image data. Here, the Adams test image data may be image data for determining whether or not the subject to be measured has scoliosis. Such an image data may be obtained by capturing, with the camera 13, the image of the subject to be measured in a state in which he/she stands in an upright posture and bends over while naturally hanging his/her arms down. That is, the back image data may include the image data and the Adams test image data in the state in which the measurement stands in the upright posture.

The image processing part 111 may extract depth frame data of the subject to be measured from the front image data, the lateral image data, and the back image data of the subject to be measured. The depth frame data may refer to photograph data including three-dimensional (3D) information which represents a depth.

The image processing part 111 may extract front depth frame data, lateral depth frame data, and back depth frame data from the front image data, the lateral image data, and the back image data. For example, the image processing part 111 may convert the color frame data of the image data into the depth frame data. The depth frame data may include subject to be measured depth frame data encompassing the subject to be measured, and background depth frame data encompassing a background other than the subject to be measured.

The image processing part 111 may remove the background depth frame data included in each of the front depth frame data, the lateral depth frame data, and the back depth frame data to extract the depth frame data of the subject to be measured. In other words, the image processing part 111 may remove the background depth frame data included in the pieces of depth frame data on the front side, the lateral side, and the back side of the subject to be measured to leave only the depth frame data on the front side, the lateral side, and the back side of the subject to be measured. Further, the image processing part 111 may synthesize the pieces of depth frame data on the front side, the lateral side, and the back side to generate a single 3D depth frame data of the subject to be measured. Thus, the pieces of depth frame data of the subject to be measured may be the 3D depth frame data.

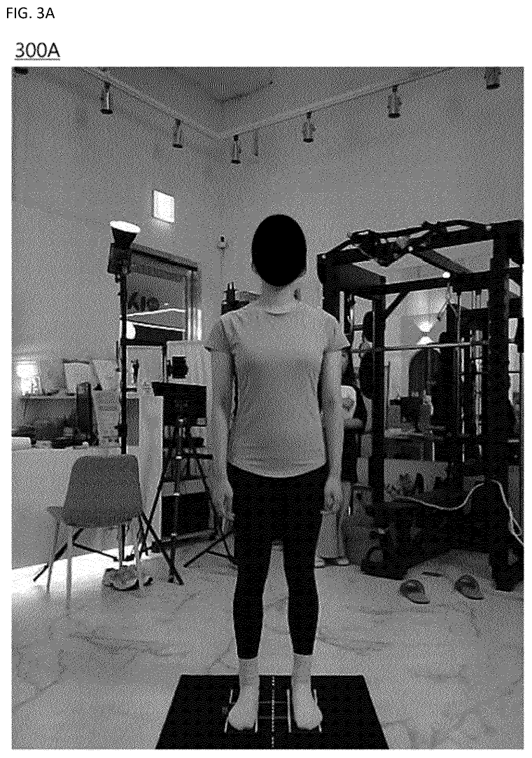

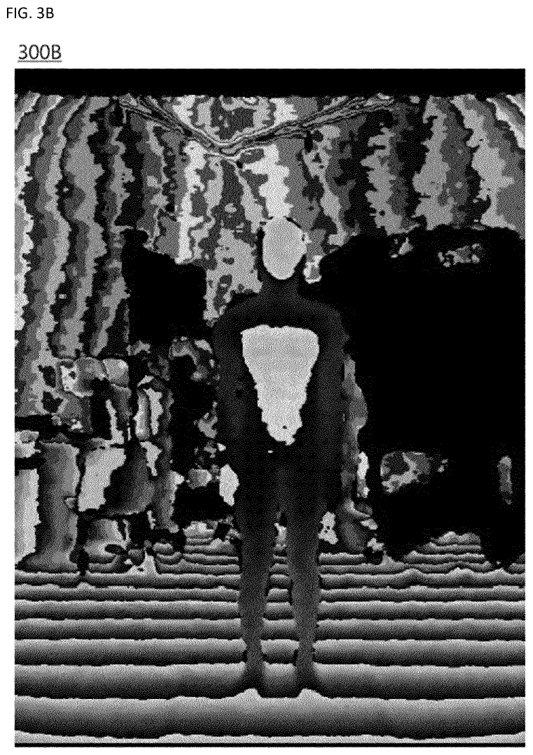

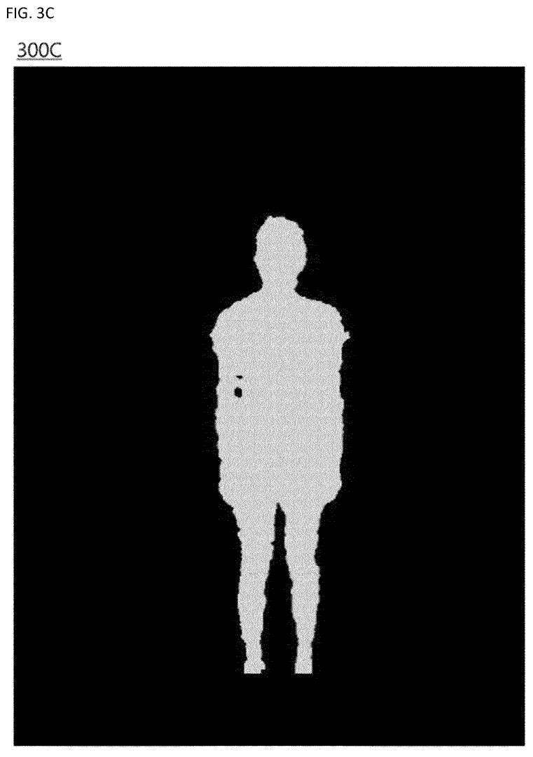

Referring to FIG. 2 and FIGS. 3A to 3C, the camera 13 may capture a front image of the body of the subject to be measured and transmit front image data 300A to the image processing part 111. The image processing part 111 may receive the front image data 300A from the camera 13 and extract front depth frame data 300B from the front image data 300A. The front depth frame data 300B may include measurement-object depth frame data encompassing a posture of the subject to be measured and the background depth frame data encompassing the background other than the subject to be measured. The image processing part 111 may obtain depth frame data 300C of the subject to be measured on the front side by removing the background depth frame data included in the front depth frame data 300B.

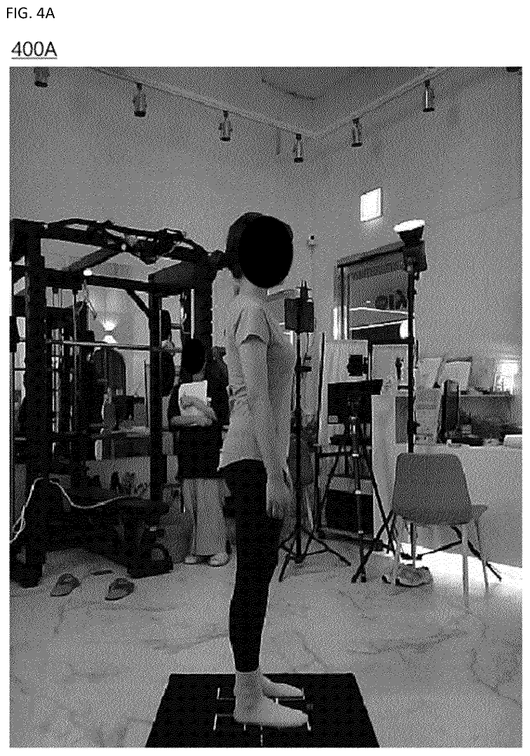

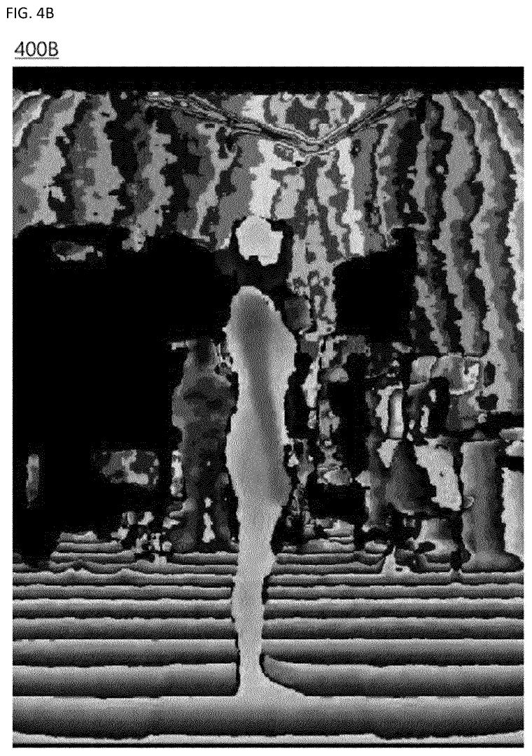

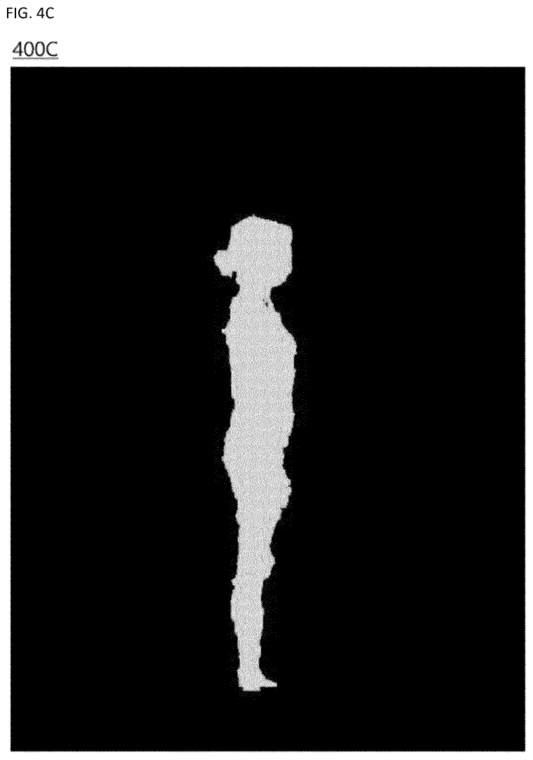

Similarly, referring to FIG. 2 and FIGS. 4A to 4C, the camera 13 may capture a lateral image of the body of the subject to be measured and transmit lateral image data 400A to the image processing part 111. The image processing part 111 may receive the lateral image data 400A and obtain, from the lateral image data 400A, lateral depth frame data 400B of the subject to be measured, which encompasses the background, and the lateral depth frame data 400C of the subject to be measured, which excludes the background.

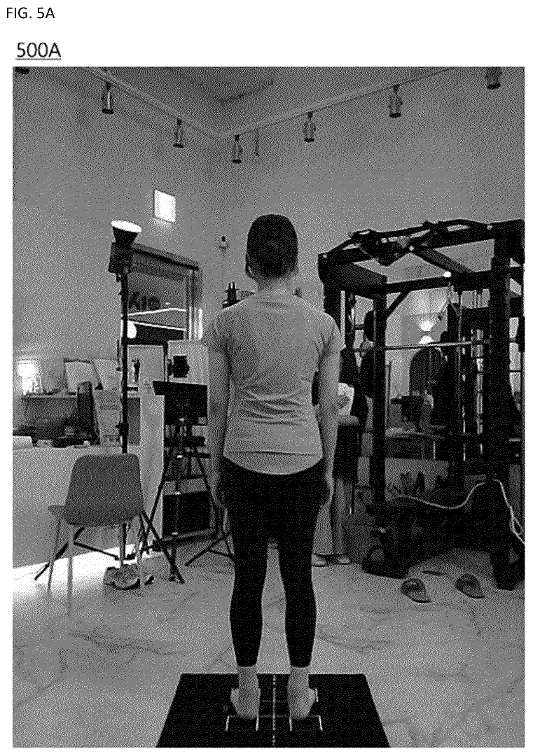

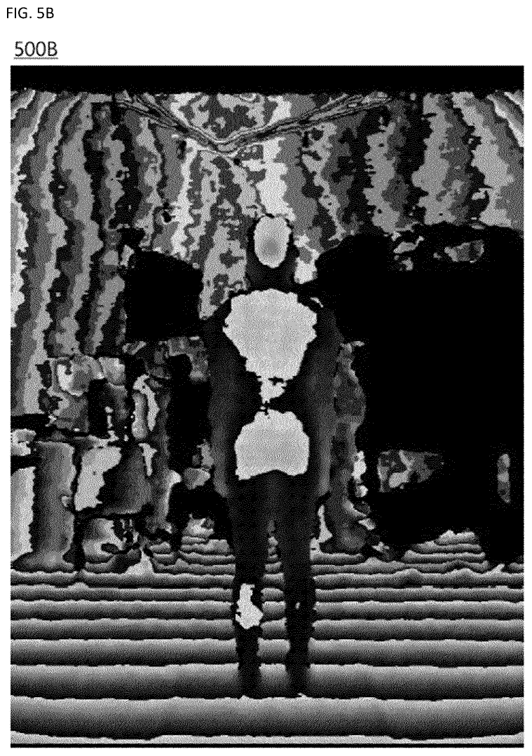

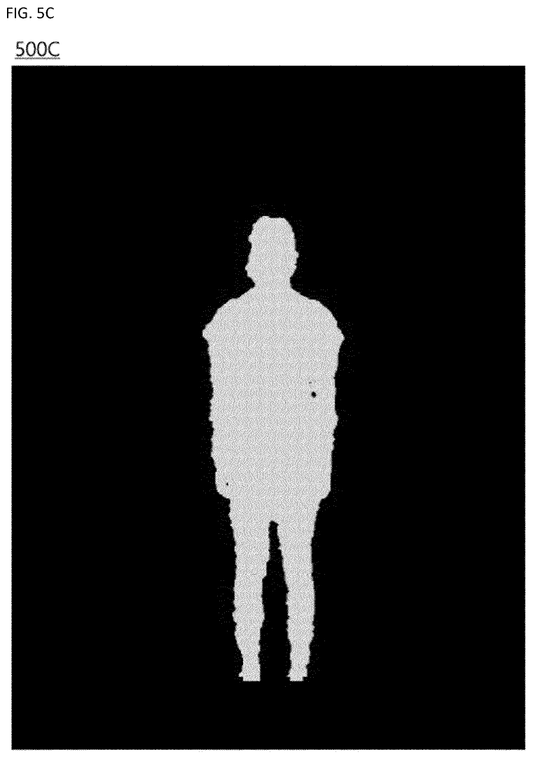

Similarly, referring to FIG. 2 and FIGS. 5A to 5C, the camera 13 may capture a back image of the body of the subject to be measured and transmit back image data 500A to the image processing part 111. The image processing part 111 may receive the back image data 500A and obtain, from the back image data 500A, back depth frame data 500B of the subject to be measured, which encompasses the background, and back depth frame data 500C of the subject to be measured, which excludes the background.

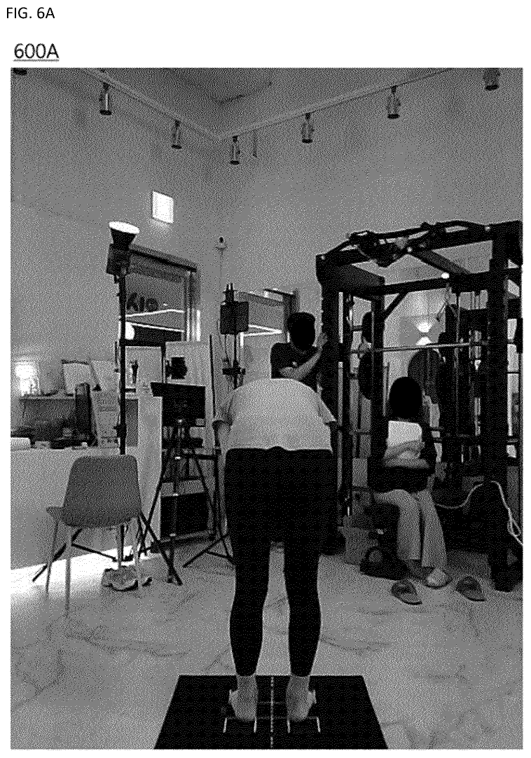

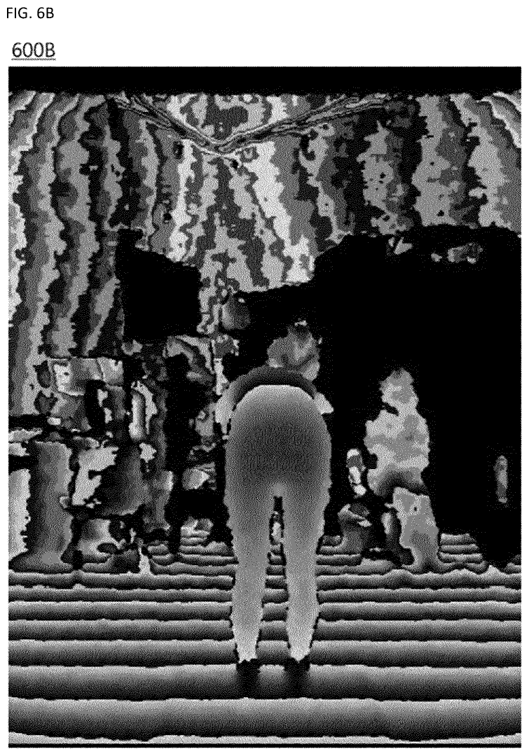

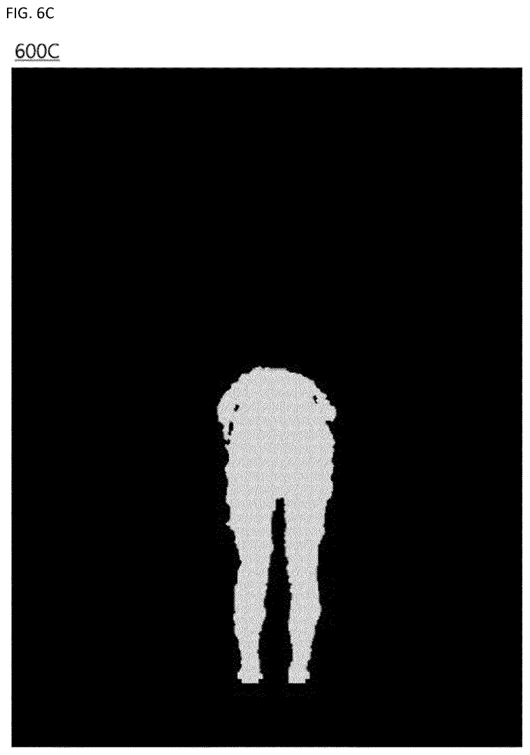

The back image data may include Adams test image data. Thus, referring to FIG. 2 and FIGS. 6A to 6C, the camera 13 may further a back image of the body of the subject to be measured and transmit Adams test image data 600A to the image processing part 111. The image processing part 111 may receive the Adams test image data 600A and obtain, from the Adams test image data 600A, Adams test depth frame data 600B of the subject to be measured, which encompasses the background, and Adams test depth frame data 600C of the subject to be measured, which excludes the background.

The skeleton point extraction part 112 may extract skeleton points of the subject to be measured based on the depth frame data of the subject to be measured obtained in the image processing part 111. The skeleton point extraction part 112 may receive the depth frame data of the subject to be measured from the image processing part 111, and compute the depth frame data of the subject to be measured to extract front skeleton point data, lateral skeleton point data, and back skeleton point data. The skeleton point extraction part 112 may transmit the front skeleton point data, the lateral skeleton point data, and the back skeleton point data to the evaluation item calculation part 113.

The skeleton point extraction part 112 may classify front body frame data, lateral body frame data, and back body frame data from the depth frame data of the measurement by a superpixel algorithm. The superpixel algorithm may be a technique which divides image data into small regions with similar features to classify them on a unit of region.

For example, the superpixel algorithm may classify the front body frame data in which a portion of the body of the subject to be measured on the front side is divided into small regions, the lateral body frame data in which a portion of the body of the subject to be measured on the lateral side is divided into small regions, and the back body frame data in which a portion of the body of the subject to be measured on the back side is divided into small regions.

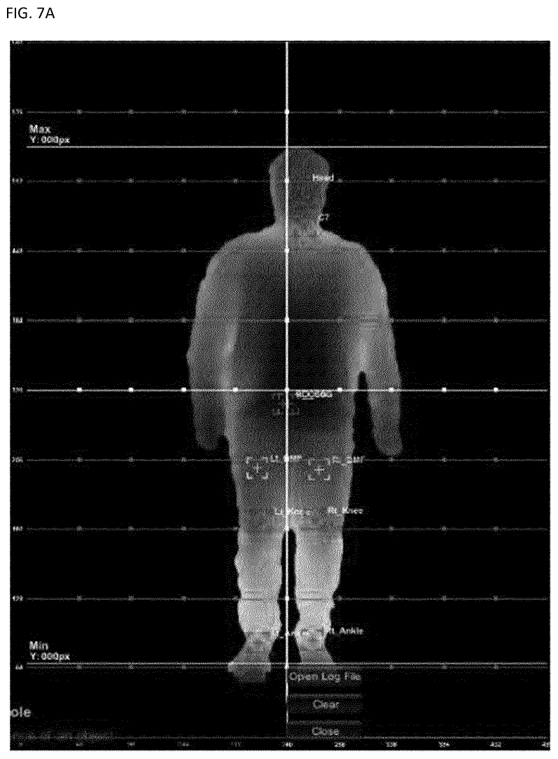

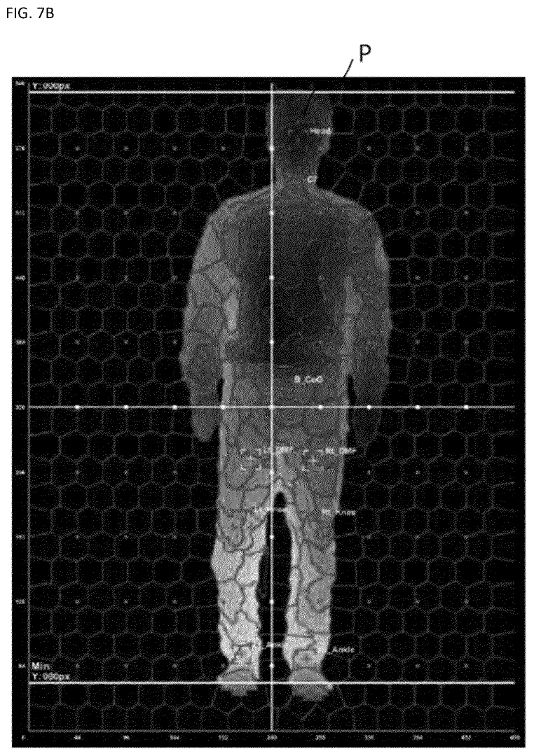

Referring to FIGS. 7A and 7B, by the superpixel algorithm, the depth frame data may be classified into small regions P each of which having similar features. The depth frame data in which the entire region thereof is classified into the small regions P on a region unit may be produced.

Further, the skeleton point extraction part 112 may extract front body portion data, lateral body portion data, and back body portion data from the front body frame data, the lateral body frame data, and the back body frame data through a convolutional neural network (CNN) operation. For example, the skeleton point extraction part 112 may perform the CNN operation on the front body frame data, the lateral body frame data, and the back body frame data with filters considering characteristics of each portion of the body to classify portions of the body on the front side, the lateral side and the back side. Thus, the skeleton point extraction part 112 may generate the front body portion data representing a front portion of the body, the lateral body portion data representing a lateral portion of the body, and the back body portion data representing a back portion of the body.

In addition, the skeleton point extraction part 112 may calculate center points included in the front body portion data, the lateral body portion data, and the back body portion data to extract the front skeleton point data, the lateral skeleton point data, and the back skeleton point data, respectively. For example, center points of body portions included in the front body portion data may be calculated to extract the front skeleton point data, center points of body portions included in the lateral body portion data may be calculated to extract the lateral skeleton point data, and center points of body portions included in the back body portion data may be calculated to extract the back skeleton point data.

According to an example embodiment of the present disclosure, the skeleton point extraction part 112 may extract 12 pieces of front skeleton point data, 24 pieces of lateral skeleton point data, and 17 pieces of back skeleton point data, respectively.

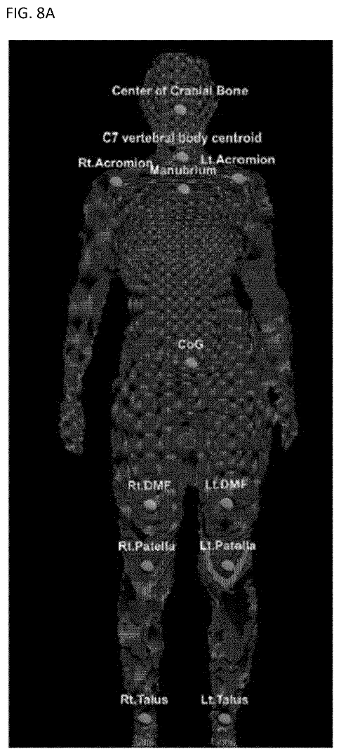

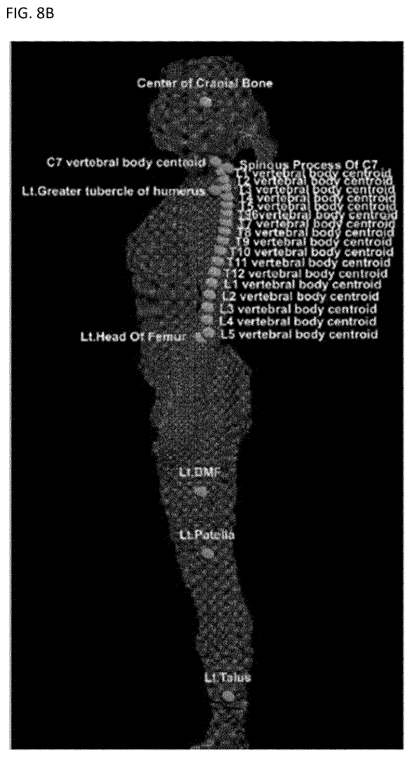

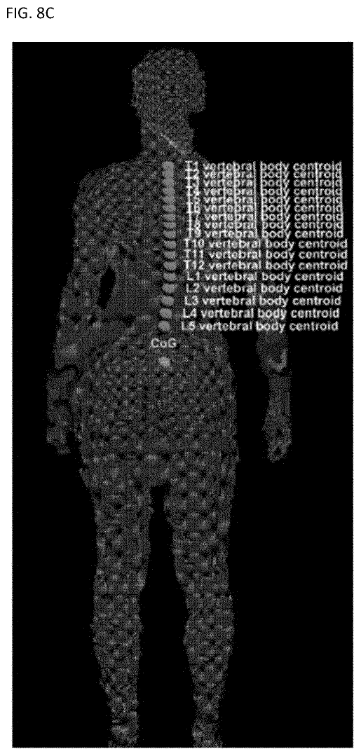

Referring to FIG. 1 and FIG. 8A, the skeleton point extraction part 112 may extract the front skeleton point data included in the depth frame data of the subject to be measured as viewed from the front side. The front skeleton point data may include first to twelfth front skeleton point data.

For example, the first front skeleton point data may have coordinates of a center of a cranial bone on X, Y, and Z axes. The second skeleton point data may have coordinates of a vertebral body centroid of a seventh cervical vertebra (C7) on the X, Y, and Z axes. The third skeleton point data may have coordinates of a left acromion portion (Lt Acromion) on the X, Y, and Z axes. The fourth skeleton point data may have coordinates of a right acromion portion (Rt Acromion) on the X, Y, and Z axes. The fifth skeleton point data may have coordinates of a middle portion of a manubrium on the X, Y, and Z axes. The sixth skeleton point data may have coordinates of a left femur (Lt DMF) on the X, Y, and Z axes. The seventh skeleton point data may have coordinates of a right femur (Rt DMF) on the X, Y, and Z axes. The eighth skeleton point data may have coordinates of a left patellar (Lt Patella) on the X, Y, and Z axes. The ninth skeleton point data may have coordinates of a right patellar (Rt Patella) on the X, Y, and Z axes. The tenth skeleton point data may have coordinates of a left talus (Lt Talus) on the X, Y, and Z axes. The eleventh skeleton point data may have coordinates of a right talus (Rt Talus) on the X, Y, and Z axes. The twelfth skeleton point data may have coordinates of a center of gravity (CoG) of the subject to be measured on the X, Y, and Z axes.

Referring to FIG. 1 and FIG. 8B, the skeleton point extraction part 112 may extract the lateral skeleton point data included in the depth frame data of the subject to be measured as viewed from the lateral side. The lateral skeleton point data may include first to twenty-fourth lateral skeleton point data.

For example, the first lateral skeleton point data may have the coordinates of the center of the cranial bone on the X, Y, and Z axes. The second lateral skeleton point data may have the coordinates of the vertebral body centroid of the seventh cervical vertebra (C7) on the X, Y, and Z axes. The third lateral skeleton point data may have coordinates of a spinous process of the seventh cervical vertebra (C7) on the X, Y, and Z axes. The fourth lateral skeleton point data may have coordinates of a greater tubercle of humerus on the X, Y, and Z axes. The fifth to sixteenth lateral skeleton point data may have coordinates of centroids of first to twelfth vertebral bodies (T1 to T12) on the X, Y, and Z axes. The seventeenth to twenty-first lateral skeleton point data may have coordinates of centroids of thirteenth to seventeenth vertebral bodies (L1 to L5) on the X, Y, and Z axes. The twenty-second lateral skeleton point data may have the coordinates of the left femur (Lt DMF) on the X, Y, and Z axes. The twenty-third lateral skeleton point data may have the coordinates of the left patellar (Lt Patellar) on the X, Y, and Z axes. The twenty-fourth lateral skeleton point data may have the coordinates of the left talus (Lt Talus) on the X, Y, and Z axes.

Referring to FIG. 1 and FIG. 8C, the skeleton point extraction part 112 may extract the back skeleton point data included in the depth frame data as viewed from the back side. The back skeleton point data may include first to seventeenth back skeleton point data.

For example, the first to twelfth back skeleton point data may have the coordinates of the centroids of the first to twelfth vertebral bodies on the X, Y, and Z axes. The thirteenth to seventeenth back skeleton point data may have the coordinates of centroids of the thirteenth to seventeenth vertebral bodies on the X, Y, and Z axes.

The skeleton point extraction part 112 may calculate the centroids included in the front body portion data, the lateral body portion data, and the back body portion data using Mathematical formula (1) below. In Mathematical formula (1), “R” may represent a set of pixels classified for each body portion. In other words, “R” may represent all pixels classified for each body portion from the depth frame data of the subject to be measured by the superpixel algorithm and the CNN operation. Thus, the skeleton point data of the subject to be measured, including the coordinates on the X, Y, and Z axes, may be extracted by Mathematical formula (1). For example, “R” may represent all pixels classified as the head of the subject to be measured from the depth frame data of the subject to be measured. The coordinates of the center of the cranial bone on the X, Y, and Z axes may be extracted by Mathematical formula (1).

$${C}_{x} = {\frac{1}{\vert R \vert} \sum x}_{( x , y , z ) ∈ R} , {C}_{y} = {\frac{1}{\vert R \vert} \sum y}_{( x , y , z ) ∈ R} , {C}_{z} = {\frac{1}{\vert R \vert} \sum z}_{( x , y , z ) ∈ R} \qquad Mathematical Formula ( 1 )$$

For reference, “C” in Mathematical formula (1) may represent the centroid.

Referring to FIG. 2, the evaluation item calculation part 113 may calculate 15 skeleton status evaluation items as listed in Table 1 below based on the extracted skeleton point data of the subject to be measured.

TABLE 1

| Section | Evaluation item | Unit | Reference |
|---|---|---|---|
| EV1 | Asymmetrical shoulder height | Degree | 0° |
| EV2 | Head posture | Degree | 11° |
| EV3 | Round shoulder | Degree | 5° |
| EV4 | Scoliosis | Degree | 0° |
| EV5 | Thoracic kyphosis | Degree | 36° |
| EV6 | Straight back syndrome | Degree | 36° |
| EV7 | Lumbar lordosis | Degree | 35° |
| EV8 | Flat back syndrome | Degree | 35° |
| EV9 | Pelvic tilt | Degree | 0° |
| EV10 | Pelvic axial rotation | Degree | 0° |
| EV11 | Pelvic obliquity | Degree | 0° |
| EV12 | HKA-angle_Lt | Degree | 0° |
| EV13 | HKA-angle_Rt | Degree | 0° |
| EV14 | Knee flexion | Degree | 0° |
| EV15 | Back knee | Degree | 0° |

In Table 1, the asymmetrical shoulder height (EV1) may be calculated from an angle of a line connecting two pieces of skeleton point data “Rt Acromion” and “Lt Acromion.” The head posture (EV2) may be calculated from an angle of a line connecting the skeleton point data “spinous process of C7” and the center of the cranial bone. The round shoulder (EV3) may be calculated from an angle in a transverse cross-section connecting two pieces of skeleton point data “Lt Greater Tubercle” and “Spinous Process of C7.” The scoliosis (EV4) may be calculated from an average value obtained by dividing the skeleton point data “T1 to L5” into seven sets of sections (T1 to T7→T2 to T8→T3 to T9) and accumulating VCM angle values thereof. The thoracic kyphosis (EV5) may be calculated from an average value obtained by dividing the skeleton point data “T1 to T12” into four sets of sections (T1 to T4→T2 to T5→T3 to T6) and accumulating VCM angle values thereof. The straight back syndrome (EV6) may be calculated from an average value obtained by dividing the skeleton point data “T1 to T12” into four sets of sections (T1 to T4→T2 to T5→T3 to T6) and accumulating VCM angle values thereof. The lumbar lordosis (EV7) may be calculated from an average value obtained by dividing the skeleton point data “L1 to L5” into three sets of sections (L1 to L3→L2 to L4→L3 to L5) and accumulating VCM angle values thereof. The flat back syndrome (EV8) may be calculated from an average value obtained by dividing the skeleton point data “L1 to L5” into three sets of sections (L1 to L3→L2 to L4→L3 to L5) and accumulating VCM angle values thereof. The pelvic tilt (EV9), the pelvic axial rotation (EV10), and the pelvic obliquity (EV11) may be calculated by inputting a total of four sheets of body surface data corresponding to the front, lateral and back sides, including Adams data, to an artificial intelligence (AI) model and receiving information about a tilt, an axial rotation, and an obliquity angle from the AI model. The HKA angle left (EV12) may be calculated by subtracting an angle defined between three points (Lt DMF, Lt Patella and Lt Talus) from 180 degrees. The HKA angle right (EV13) may be calculated by subtracting an angle defined between three points (Rt DMF, Rt Patella and Rt Talus) from 180 degrees. The knee flexion (EV14) may be calculated by subtracting an angle defined between three points in a sagittal plane (Lt DMF, Lt Patella and Lt Talus) from 180 degrees. The back knee (EV15) may be calculated by subtracting an angle defined between three points in the sagittal plane (Lt DMF, Lt Patella and Lt Talus) from 180 degrees.

The evaluation results obtained by the evaluation item calculation part 113 may be transmitted to the skeleton simulation part 114, the muscular condition diagnosis part 115, and the evaluation result comparison part 117.

The skeleton simulation part 114 generates simulation data for displaying a skeletal morphology of the subject to be measured according to 53 pieces of skeleton point data extracted as above and the evaluation results obtained by the evaluation item calculation part 113.

The muscular condition diagnosis part 115 estimates that, when a value of each of the evaluation items is less than or greater than a predetermined value, some muscles are in a tightened state, and other muscles are in a weakened state. Levels of the tightened state and the weakened state of individual muscles may be applied as a risk rate calculated by the risk rate prediction part 118.

The muscular condition simulation part 116 generates simulation data for displaying the muscular condition of the subject to be measured according to a diagnosis result of the muscular condition diagnosis part 115.

The evaluation result comparison part 117 compares values of the calculation result obtained by the evaluation item calculation part 113 with normal state data of the normal state data part 124. Based on the comparison result and the evaluation results relating to the evaluation items, the muscular condition diagnosis part 115 diagnoses weakened muscles and tightened muscles, which are caused due to deformed skeletons, among 44 muscles (G01 to G44) of the subject to be measured as listed in Table 2 below.

TABLE 2

| No | Muscle Name |
|---|---|
| G01 | Middle scalenus |
| G02 | Sternocleidomastoid muscle |
| G03 | Longus colli muscle |
| G04 | longus capitis muscle |
| G05 | psoas minor muscle |
| G06 | iliopsoas muscle |
| G07 | iliacus muscle |
| G08 | sartorius muscle |
| G09 | erector spinae muscle |
| G10 | gluteus medius muscle |
| G11 | gluteus minimus |
| G12 | Tensor fasciae latae |
| G13 | Iliotibial band |
| G14 | Quadratus Lumborum |
| G15 | External oblique muscle |
| G16 | Internal oblique muscle |
| G17 | Rectus abdominis |
| G18 | Transversus abdominis muscle |
| G19 | Rectus femoris muscle |
| G20 | Vastus intermedius, cruraeus |
| G21 | Vastus lateralis, vastus externus |
| G22 | Gastrocnemius muscle |
| G23 | Vastus medialis |
| G24 | Gracilis muscle |
| G25 | Pectineus muscle |
| G26 | Abdominal muscle |
| G27 | Adductor longus muscle |
| G28 | Adductor magnus |
| G29 | Suboccipital muscle |
| G30 | Levator scapulae |
| G31 | Collarbone pectoralis major muscle |
| G32 | sternum pectoralis major muscle |
| G33 | Pectoralis minor muscle |
| G34 | Upper trapezius |
| G35 | Middle trapezius |
| G36 | Lower trapezius |
| G37 | Latissimus dorsi muscle |
| G38 | Rhomboideus major |
| G39 | Rhomboideus minor |
| G40 | Biceps femoris muscle |
| G41 | Semimembranosus muscle |
| G42 | Semitendinosus |
| G43 | Gluteus maximus |
| G44 | Popliteus muscle |

The risk rate prediction part 118 predicts the risk rate according to the comparison result obtained by the evaluation result comparison part 117 to provide prediction information. That is, the risk rate prediction part 118 calculates the risk rate by using a normal probability distribution as expressed in Mathematical formula (2) as a method of evaluating a risk rate for each of 11 evaluation items. A standard deviation and an average of the evaluation items may be calculated with data gathered through accumulated data and big data. The risk rate of the evaluation result is expressed as a percentage (%).

$$\frac{1}{σ \sqrt{2 π}} {e}^{- \frac{1}{2} {( \frac{x - μ}{σ} )}^{2}} \qquad [ Mathematical Formula 2 ]$$

In Mathematical formula (2), “μ” represents the average of the evaluation items, “σ” represents the standard deviation of the evaluation items, and “x” represents the values of the evaluation items.

The user interface part 119 synthesizes skeleton simulation data, muscular condition simulation data and risk rate prediction data and provides the synthesized data to the display 31 in the form of graphics and letters that is easy for the subject to be measured to understand.

Referring to FIG. 2, the reference frame data part 125 may store reference frame data provided from the service server 20, and the normal state data part 124 may store normal state standard data relating to the evaluation items. The user data transmission part 122 transmits the measurement data and the evaluation results relating to the subject to be measured to the service server 20 via the server communication part 121. The reference data update part 123 communicates with the service server 20 via the server communication part 121 and updates pieces of data in the reference frame data part 125 and the normal state data part 124 with new reference data provided from the service server 20 to improve measurement accuracy.

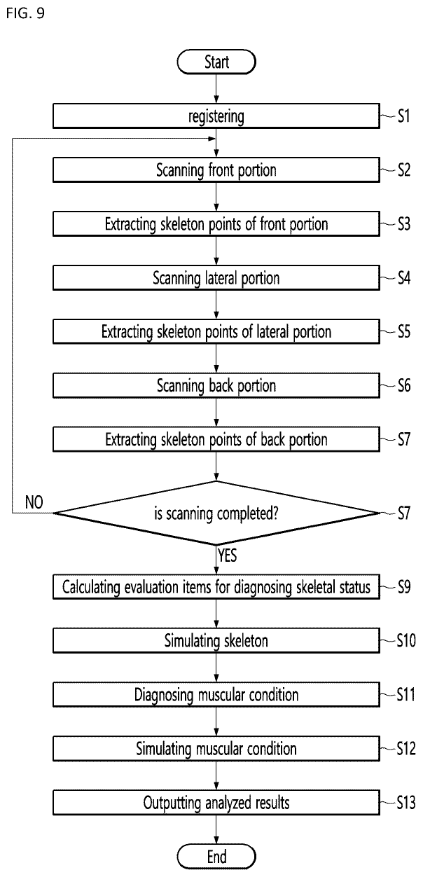

FIG. 9 is a flowchart for explaining a musculoskeletal analysis procedure according to an example embodiment of the present disclosure.

As illustrated in FIG. 9, in the musculoskeletal analysis procedure according to an example embodiment of the present disclosure, when the musculoskeletal status analysis system 10 is turned on, a musculoskeletal analysis program is executed to display a main screen. In a case in which a subject to be measured is a new user, he/she selects a menu on the main screen to establish his/her own database. In a case in which the subject to be measured is a previously-registered user, he/she accesses his/her user database which has been previously registered (Operation S1). Items such as at least a user's name, height, age, e-mail address, gender, previous measurement information, measurement date, or the like, is provided in the user database and respective information is recorded in the user database.

After a user registration process is completed, an image of a front portion of the subject to be measured located on the footplate 12 is captured by the camera 13 at a scan rate of about 3 seconds, and data on the image of the front portion is analyzed to extract the front skeleton point data (Operations S2 and S3). The front skeleton point data has the 12 skeleton points.

After the image of the front portion of the subject to be measured is captured, an image of a lateral portion of the subject to be measured located on the footplate 12 is captured by the camera 13 at a scan rate of about 3 seconds, and data on the image of the lateral portion is analyzed to extract the lateral skeleton point data (Operations S4 and S5). The lateral skeleton point data has the 24 skeleton points.

After the image of the lateral portion of the subject to be measured is captured, an image of a back portion of the subject to be measured located on the footplate 12 is captured by the camera 13 at a scan rate of about 3 seconds, and data on the image of the back portion is analyzed to extract the back skeleton point data (Operations S6 and S7). The back skeleton point data has the 17 skeleton points. A procedure of extracting respective pieces of skeleton point data is the same as described above.

After all the capturing is completed, the pieces of skeleton point data extracted as above are synthesized, 11 evaluation items for evaluating the skeletal morphology are calculated, and the simulation data for displaying the skeletal morphology of the subject to be measured is generated based on calculated values (Operations S8 to S10). In Operation S9 of calculating the evaluation items, evaluation items for diagnosing the skeletal status of the subject to be measured from the skeleton points extracted as described with reference to Table 2 above are calculated.

In Operation S11 of diagnosing the muscular condition, as described above, 44 muscles of the subject to be measured are diagnosed based on the evaluation results of the evaluation items and the pieces of skeleton point data. The 44 muscles are classified into weakened muscle(s) and tightened muscle(s) which are affected by a deformed skeleton, and normal muscle(s). With such diagnosis results, an operator may recommend a muscle strengthening exercise to the subject to be measured so as to strengthen the weakened muscle(s), and may recommend a stretching exercise to the subject to be measured so as to relax the tightened muscle(s).

In Operation S12 of simulating the muscular condition, simulation data for displaying the conditions of the 44 muscles diagnosed as above is generated. In Operation S13 of outputting an analysis result, information such as skeleton simulation data, muscular condition simulation data, a risk rate and the like are provided on the display 31 so that the subject to be measured may check his/her own musculoskeletal status.

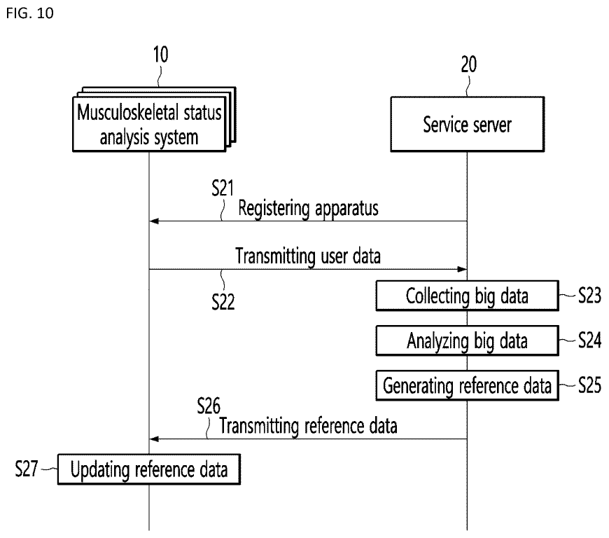

FIG. 10 is a flowchart for explaining a procedure of updating reference data through a big data analysis according to an example embodiment of the present disclosure.

Referring to FIG. 10, when the musculoskeletal status analysis system 10 is installed in healthcare centers or the like across the country, it is registered in the service server 20 via the network 21 (S21). The service server 20 manages information about the registered apparatuses, collects various pieces of measurement information about various subjects to be measured from the musculoskeletal status analysis systems 10 and stores the same in a database (S22 and S23). In some instances, various pieces of necessary data may be collected using a big data collection technique via a network.

The service server 20 analyzes the pieces of collected musculoskeletal information using the big data analysis technique and generates the reference data for musculoskeletal analysis (S24 and S25). In an example embodiment of the present disclosure, examples of the reference data may include the reference frame data and the normal state data relating to the evaluation items.

Subsequently, the generated reference data is transmitted to the registered apparatuses. With this configuration, each apparatus may update the reference data, which makes it possible to more accurately perform the musculoskeletal analysis (S26 and S27).

Although the technical spirit of the present disclosure has been described using embodiments illustrated in the accompanying drawings, it should be noted that various modifications, and equivalent variations can be devised by those skilled in the art to which the present disclosure pertains without departing from the technical spirit and scope of the present disclosure.

What is claimed is:

1. An image data-based musculoskeletal status analysis system comprising:
an image processing part configured to receive front image data, lateral image data, and back image data of a subject to be measured, and extract pieces of depth frame data of the subject to be measured from each of the front image data, the lateral image data, and the back image data;
a skeleton point extraction part configured to receive the pieces of depth frame data of the subject to be measured from the image processing part and calculate the pieces of depth frame data of the subject to be measured to extract pieces of skeleton point data;
an evaluation item calculation part configured to calculate values of evaluation items for evaluating a skeletal condition from the pieces of skeleton point data of the subject to be measured extracted by the skeleton point extraction part; and
a muscular condition diagnosis part configured to diagnose a muscular condition by comparing the values of the evaluation items calculated by the evaluation item calculation part with data in a normal state.

2. The image data-based musculoskeletal status analysis system of claim 1, wherein the image processing part is configured to:
extract front depth frame data, lateral depth frame data, and back depth frame data from the front image data, the lateral image data, and the back image data, respectively; and
remove background depth frame data included in each of the front depth frame data, the lateral depth frame data, and the back depth frame data to extract the depth frame data of the subject to be measured.

3. The image data-based musculoskeletal status analysis system of claim 2, wherein the skeleton point extraction part is configured to classify front body frame data, lateral body frame data, and back body frame data from the depth frame data of the subject to be measured through a superpixel algorithm.

4. The image data-based musculoskeletal status analysis system of claim 3, wherein the skeleton point extraction part is configured to extract front body portion data, lateral body portion data and back body portion data from the front body frame data, the lateral body frame data and the back body frame data through a convolutional neural network (CNN) operation.

5. The image data-based musculoskeletal status analysis system of claim 4, wherein the skeleton point extraction part is configured to calculate center points included in the front body portion data, the lateral body portion data, and the back body portion data to extract front skeleton point data, lateral skeleton point data, and back skeleton point data, respectively.

6. The image data-based musculoskeletal status analysis system of claim 5, wherein the skeleton point extraction part is configured to extract the front skeleton point data, the lateral skeleton point data, and the back skeleton point data which include coordinates on X, Y, and Z axes through
$${C}_{x} = {\frac{1}{\vert R \vert} \sum x}_{( x , y , z ) ∈ R} , {C}_{y} = {\frac{1}{\vert R \vert} \sum y}_{( x , y , z ) ∈ R} , {C}_{z} = {\frac{1}{\vert R \vert} \sum z}_{( x , y , z ) ∈ R}$$
(where C represents the center point, and R represents all pixels classified by a body portion of the subject to be measured from the depth frame data of the subject to be measured).

7. The image data-based musculoskeletal status analysis system of claim 6, wherein the skeleton point extraction part is configured to extract:
the front skeleton point data including coordinates of a center of a cranial bone on the X, Y, and Z axes, coordinates of a vertebral body centroid of a seventh cervical vertebra on the X, Y, and Z axes, coordinates of a left acromion portion on the X, Y, and Z axes, coordinates of a right acromion portion on the X, Y, and Z axes, coordinates of a middle portion of a manubrium on the X, Y, and Z axes, coordinates of a left femur on the X, Y, and Z axes, coordinates of a right femur on the X, Y, and Z axes, coordinates of a left patellar on the X, Y, and Z axes, coordinates of a right patellar on the X, Y, and Z axes, coordinates of a left talus on the X, Y, and Z axes, coordinates of a right talus on the X, Y, and Z axes, and coordinates of a center of gravity (CoG) of the subject to be measured on the X, Y, and Z axes;
the lateral skeleton point data including the coordinates of the center of the cranial bone on the X, Y, and Z axes, the coordinates of the vertebral body centroid of the seventh cervical vertebra on the X, Y, and Z axes, coordinates of a spinous process of the seventh cervical vertebra on the X, Y, and Z axes, coordinates of a greater tubercle of humerus on the X, Y, and Z axes, coordinates of centroids of first to twelfth vertebral bodies on the X, Y, and Z axes, coordinates of centroids of thirteenth to seventeenth vertebral bodies on the X, Y, and Z axes, the coordinates of the left femur on the X, Y, and Z axes, the coordinates of the left patellar on the X, Y, and Z axes, and the coordinates of the left talus on the X, Y, and Z axes; and
the back skeleton point data including the coordinates of the centroids of the first to twelfth vertebral bodies on the X, Y, and Z axes, and the coordinates of centroids of the thirteenth to seventeenth vertebral bodies on the X, Y, and Z axes.

8. The image data-based musculoskeletal status analysis system of claim 7, wherein the evaluation item calculation part is configured to calculate evaluation items including an asymmetrical shoulder height, a head posture, a round shoulder, a scoliosis, a thoracic kyphosis, a straight back syndrome, a lumbar lordosis, a flat back syndrome, a pelvic tilt, a pelvic axial rotation, a pelvic obliquity, a left Hip-Knee-Ankle (HKA) angle, a right HKA angle, a knee flexion, and a back knee.

9. The image data-based musculoskeletal status analysis system of claim 1, further comprising:
a skeleton simulation part configured to generate data for displaying a skeletal morphology of the subject to be measured based on the pieces of skeleton point data of the subject to be measured and the values of the evaluation items;
an evaluation result comparison part configured to compare result values calculated by the evaluation item calculation part with normal state data of a normal state data part;
a risk rate prediction part configured to predict a risk rate based on a comparison result obtained by the evaluation result comparison part and provide prediction information; and
a user interface part configured to provide skeleton simulation data obtained by the skeleton simulation part, data on the muscular condition obtained by the muscular condition diagnosis part, and the prediction information obtained by the risk rate prediction part to a user via a display part or a printer.
